(19) United States
(12) Plant Patent Application Publication
(10) Pub. No.: US 2025/0255202 P1
(43) Pub. Date: Aug. 7, 2025

(54) BEGONIA PLANT NAMED 'BEYZ0004'

(50) ;

(71) Applicant: SYNGENTA CROP PROTECTION AG, Basel (CH)

(72) Inventor: Cathy Knoblauch, Whitecourt (CA)

(73) Assignee: SYNGENTA CROP PROTECTION AG, Basel (CH)

(21) Appl. No.: 18/445,790

(22) Filed: Feb. 1, 2024

(51) Int. Cl. A01H 6/18

(52) U.S. Cl. CPC A01H 6/185
USPC PLT/434

(57) ABSTRACT

A new Begonia plant named ‘BEYZ0004’ distinguished by its compact and rounded symmetrical habit, and freely produced basal branching. It is further characterized by its continuous natural flowering habit and deep mahogany glossy foliage and exhibits deep red double flowers.

Latin name of the genus and species of the plant claimed: Begonia hybrida.

Varietal denomination: ‘BEYZ0004’

### BACKGROUND OF THE NEW PLANT

The present invention comprises a new Begonia plant, botanically known as Begonia hybrida and hereinafter referred to by the variety name ‘Adora Velvet Red’.

‘Adora Velvet Red’ is a product of a planned breeding program in Whitecourt, Alberta, Canada. The new cultivar ‘Adora Velvet Red’ has compact and rounded symmetrical habit, prolific and natural branching. It is further characterized by its continuous natural flowering habit and deep mahogany glossy foliage and exhibits deep red double flowers.

The new Begonia cultivar, ‘Adora Velvet Red’, is the result of a planned pollination and was made in Whitecourt, Alberta, Canada at the inventor's nursery in the month of September in 2018. The female parent of the new cultivar is the unnamed red seedling of the inventor's proprietary selections, unpatented, characterized by its large red flowers and leaves. ‘Adora Velvet Red’ differs from the female parent in having smaller flowers and leaves.

The male parent of the new cultivar is the unnamed seedling of the inventor's proprietary selection, unpatented. ‘Adora Velvet Red’ differs from the male parent in having larger flowers and leaves, and an overall larger habit than the male parent.

The resulting seed was sown in Whitecourt, Alberta, Canada in a controlled greenhouse environment in 2018 and ‘Adora Velvet Red’ was selected as one flowering plant within the progeny of the stated cross in May 2019.

The first act of asexual reproduction of ‘Adora Velvet Red’ was accomplished when vegetative tip cuttings were propagated from the initial selection in May 2019, in Whitecourt, Alberta Canada.

### BRIEF SUMMARY OF INVENTION

Plants of the new Begonia have not been observed under all possible combinations of environmental conditions and cultural practices. The phenotype may vary somewhat with variations in environmental conditions such as temperature and light intensity, without, however, any variance in genotype.

A Plant Breeder's Right for this cultivar has not yet been applied for. ‘Adora Velvet Red’ has not been made publicly available prior to the effective filing date of this application, notwithstanding any disclosure that may have been made less than one year prior to the effective filing date of this application by the inventor or another who obtained directly from the inventor.

The following traits have been repeatedly observed and are determined to be basic characteristics of the new variety. The combination of these characteristics distinguishes this Begonia as a new and distinct variety.

### BRIEF DESCRIPTION OF THE PHOTOGRAPHS

The accompanying photographic drawings show typical flower and foliage characteristics of ‘Adora Velvet Red’ with colors being as true as possible with an illustration of this type.

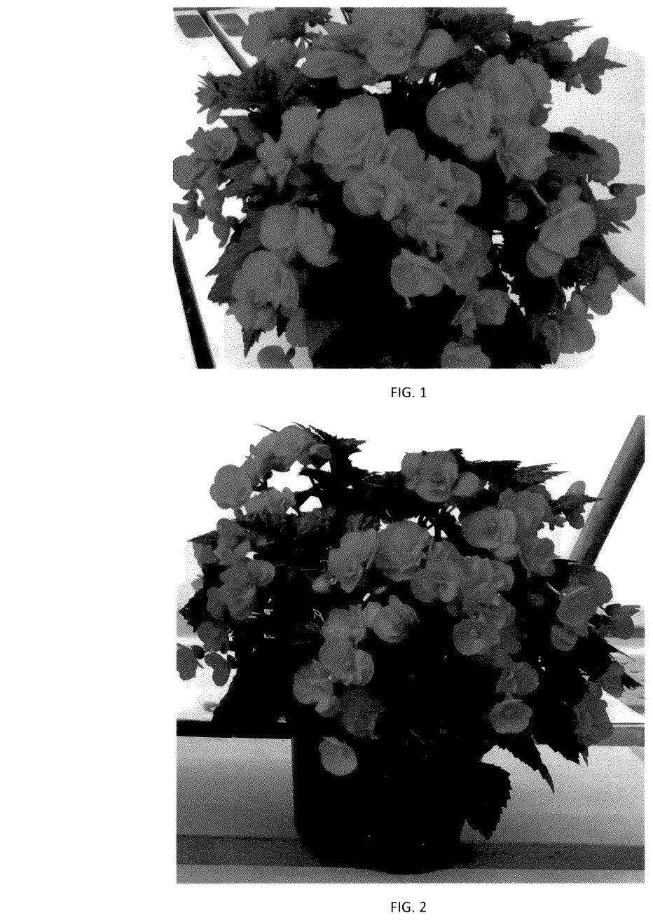

The photographic drawings show in FIG. 1 a close view of an inflorescence taken in September 2019 in Whitecourt, Alberta, Canada, and in FIG. 2 a whole flowering plant taken in September 2019 in Whitecourt, Alberta, Canada.

### DETAILED BOTANICAL DESCRIPTION

The plant descriptions and measurements were taken in July 2023 in Whitecourt, Alberta, Canada. The plants were approximately 6 months old and were growing in 8 inch pots, one plant per pot, in a greenhouse.

Color references are made to The Royal Horticultural Society Colour Chart (R.H.S.) 2001.

TABLE 1
DIFFERENCES BETWEEN THE NEW VARIETY
AND A MOST SIMILAR VARIETY

| | ‘ADORA VELVET RED’ | ‘MOCHA RED’ |
|---|---|---|
| Flower and leaf size: | Smaller | Larger |
| Foliage: | Glabrous | Pubescent |
| Lateral and basal branching: | More prolific | Less prolific |

### Traits:

1. ‘Adora Velvet Red’ has a symmetrical mounded and compact habit.
2. ‘Adora Velvet Red’ achieves a height of 25 cm and a width of 25-30 cm in one season grown in a one gallon pot.
3. ‘Adora Velvet Red’ blooms naturally and continuously from spring until fall.
4. ‘Adora Velvet Red’ exhibits prolific basal and lateral branching.
5. ‘Adora Velvet Red’ exhibits fully double flowers that are deep red in color.
6. ‘Adora Velvet Red’ exhibits dark mahogany foliage with a smooth glossy surface.
Propagation:
   Type.—Vegetative tip cuttings.
   Time to initiate roots, summer and winter.—About 15 days summer, about 20 days winter.
   Time to produce a rooted young plant, summer.—About 35 days.
   Time to produce a rooted young plant, winter.—About 45 days.
   Root description.—Medium thick, fibrous.
   Rooting habit.—Moderately freely branching.
Plant description:
   Plant and growth habit.—Upright mounded and compact.
   Plant height, soil level to top of foliar plane.—About 18 cm.
   Plant height, soil level to top of floral plane.—About 20 cm.
   Plant width.—About 30 cm.
   Lateral branch description.—Upright freely branched.
   Length.—About 15 cm.
   Diameter.—About 5-7 mm.
   Internode length.—About 3-5 cm.
   Strength.—Strong.
   Aspect.—Symmetrical.
   Texture and luster.—Smooth, glossy.
   Color, developing.—About RHS 177A.
   Color, developed.—About RHS 199A.
Leaf description:
   Arrangement.—Alternate.
   Length.—About 9 cm.
   Width.—About 5 cm.
   Shape.—Cordate, asymmetrical.
   Apex.—Acuminate.
   Base.—Cordate.
   Margin.—Irregularly dentate.
   Texture and luster, upper and lower surfaces.—Upper: glossy with sparse colorless hairs, lower: sparsely pubescent with colorless hairs along veins.
   Venation pattern.—Palmate.
   Developing leaves, upper surface.—Palmate.
   Developing leaves, lower surface.—Palmate.
   Fully expanded leaves, upper surface.—Palmate.
   Fully expanded leaves, lower surface.—Palmate.
Petioles:
   Length.—About 2.5-3 cm.
   Diameter.—About 3 mm.
   Texture and luster, upper and lower surfaces.—Sparsely pubescent, matte both upper and lower.
   Strength.—Medium.
   Color, upper surface.—Nearest RHS 184A.
   Color, lower surface.—Nearest RHS 184A.
Stipules:
   Quantity per leaf.—2 persistent.
   Length.—About 8 mm.
   Width.—About 6 mm.
   Shape.—Triangular.
   Apex.—Sharply acute.
   Base.—Truncate.
   Margins.—Ciliate.
   Color, upper and lower surfaces.—Nearest RHS 184A.
Flower description:
   Flowering habit.—Continuous.
   Fragrance.—None observed.
   Natural flowering season.—Spring to fall.
   Flower longevity.—About 30 days.
   Inflorescence height (including peduncle).—12 cm.
   Flower buds, female flowers.—.
   Length.—About 1.5 cm.
   Diameter, flattened.—About 2 cm.
   Shape.—Flattened ovoid.
   Texture and luster.—Smooth, matte.
   Color, upper surface.—Nearest RHS 45C.
   Color, lower surface.—Nearest RHS 45C.
   Flower buds, male flowers.—.
   Length.—About 4 cm.
   Diameter, flattened.—About 4 cm.
   Shape.—Ovoid.
   Texture and luster.—Smooth, matte.
   Color, upper surface.—Nearest RHS 45B.
   Color, lower surface.—Nearest RHS 45C.
   Female flowers size.—About 5 cm.
   Depth.—About 5 cm.
Tepals:
   Quantity per flower and arrangement.—5 arranged in a single whorl.
   Length, inner tepals.—About 2.5 cm.
   Length, outer tepals.—None.
   Width, inner tepals.—About 2.5 cm.
   Width, outer tepals.—None.
   Shape, inner tepals.—Broadly obovate.
   Shape, outer tepals.—None.
   Apex, inner tepals.—Very broadly obovate.
   Apex, outer tepals.—None.
   Base, inner and outer tepals.—Broadly Obovate.
   Margin, inner and outer tepals.—Entire.
   Texture and luster, inner and outer tepals, upper surface.—Smooth, matte.
   Texture and luster, inner and outer tepals, lower surface.—Smooth, matte.
   Color, inner tepals.—.
   When opening, upper surface.—Nearest RHS 45B.
   When opening, lower surface.—Nearest RHS 45B.
   Fully opened, upper surface.—Nearest RHS 45C.
   Fully opened, lower surface.—Nearest RHS 45C.
   Color, outer tepals.—.
   When opening, upper surface.—None.
   When opening, lower surface.—None.
   Fully opened, upper surface.—None.
   Fully opened, lower surface.—None.
   Tepaloids.—None observed on female flowers.
   Male flowers.—.
   Size.—About 5 cm.
   Depth.—About 4 cm.
Tepals:
   Quantity per flower and arrangement.—20-25 arranged in about 4 whorls.
   Length, inner tepals.—About 1.5 cm.
   Length, outer tepals.—About 2 cm.
   Width, inner tepals.—About 1.3 cm.
   Width, outer tepals.—About 2.2 cm.
   Shape, inner tepals.—Broadly obovate.
   Shape, outer tepals.—Broadly ovate.
   Apex, inner and outer tepals.—Very broadly ovate.
   Base, inner and outer tepals.—Broadly ovate.
   Margin, inner and outer tepals.—Entire, smooth.
   Texture and luster, inner and outer tepals, upper surface.—Smooth, matte.
   Texture and luster, inner and outer tepals, lower surface.—Smooth, matte.
   Color, inner tepals.—.
   When opening, upper surface.—About RHS 45C.
   When opening, lower surface.—About RHS 45B.
   Fully opened, upper and lower surfaces.—About RHS 45B.
   Color, outer tepals.—.
   When opening, upper surface.—About RHS 45C.
   When opening, lower surface.—About RHS 45C.
   Fully opened, upper surface: About RHS 45B.
   Fully opened, lower surface:About RHS 45B.
   Tepaloids: None observed on male flowers.
Peduncles:
   Length.—About 3.5 cm.
   Diameter.—About 2 mm.
   Angle.—Mostly upright and outward facing.
   Strength.—Moderately strong, wiry.
   Texture and luster.—Smooth, glossy.
   Color, upper and lower surfaces.—Upper, about RHS 35A, lower about RHS 182B.
Pedicels, female flowers:
   Length.—About 1.7 cm.
   Diameter.—About 1.5 mm.
   Angle.—About 45 degrees.
   Strength.—Moderately strong.
   Texture and luster.—Smooth, glossy.
   Color, upper surface.—About RHS 35A.
   Color, lower surface.—About RHS 182B.
Pedicels, male flowers:
   Length.—About 2 mm.
   Diameter.—About 2 mm.
   Angle.—Upright, about 360 degrees.
   Strength.—Moderately strong, wiry.
   Texture and luster.—Smooth, glossy.
   Color, upper surface.—About RHS 35A.
   Color, lower surface.—About RHS 182B.
Flower bracts:
   Quantity per flower and arrangement.—2.
   Length.—About 1 cm.
   Width.—About 1 cm.
   Shape.—Ovate.
   Apex.—Very broadly ovate.
   Base.—Ovate.
   Margin.—Entire, smooth.
   Texture and luster, upper and lower surfaces.—Smooth, glossy.
   Color, upper and lower surfaces.—Nearest RHS 172D.
Reproductive organs:
   Androecium.—.
   Stamen quantity per flower.—None observed.
   Filament length.—None observed.
   Filament color.—None.
   Anther size.—None.
   Anther shape.—None.
   Anther color.—None.
   Amount of pollen.—None.
   Pollen color.—None.
   Gynoecium.—.
   Quantity per flower.—One.
   Pistil length.—About 1.5 cm.
   Stigma diameter.—About 1 mm.
   Stigma shape.—Bifurcated, loose spiral.
   Stigma color.—About RHS 11B to RHS 12C.
   Style length.—About 1 cm.
   Style color.—About RHS 145D.
   Ovary color.—About RHS 153A.
   Seeds and fruits.—None observed.
Pathogen & pest resistance: To date, resistance to pathogens and pests common to Begonia plants has not been observed on plants of the new Begonia.
Temperature tolerance: Plants of the new Begonia have been observed to tolerate high temperatures of about 30° C. and to be suitable for USDA Hardiness Zones 7 to 9.

What is claimed is:

1. A new and distinct variety of Begonia plant named ‘BEYZ0004’ substantially as illustrated and described herein.
